(19) United States
(12) Patent Application Publication
(10) Pub. No.: US 2009/0048332 A1
(43) Pub. Date: Feb. 19, 2009

(54) Natural Novel Antioxidants

(75) Inventors: Muhammad Iqbal Choudhary, Karachi (PK); Sajjad Ali, Karachi (PK); Vinitha M Thadani, Kandy (LK); Veranja Karunaratne, Kandy (LK)

Correspondence Address: SARFARAZ K. NIAZI, 20 RIVERSIDE DRIVE, DEERFIELD, IL, 60015, US

(73) Assignees: HEJ Research Insitute, Karachi (PK); University of Peradeniya, Peradeniya (PK)

(21) Appl. No.: 11/838,567

(22) Filed: Aug. 14, 2007

(51) Int. Cl. A61K 31/335; A61K 31/19; A61P 35/00; A61P 39/00

(52) U.S. Cl. 514/450; 514/568

(57) ABSTRACT

New antioxidants derived from lichen extracts are reported; lecanoric acid, erythrin, sekikaic acid, and lobaric acid were reported as potent natural antioxidants for the treatment of disease and protection of products from the effect of oxidizing components.

### BACKGROUND OF THE INVENTION

Plant foods, such as fruits, vegetables, and whole grains contain many components that are beneficial to human health. Research supports that some of these foods, as part of an overall healthful diet, have the potential to delay the onset of many age-related diseases. These observations have led to continuing research aimed at identifying specific bioactive components in foods, such as antioxidants, which may be responsible for improving and maintaining health. Recent developments in medicine point to the involvement of free radicals in many human diseases. Thus, free radicals play an important role in carcinogenesis through their involvement in breaking of DNA strands [Pathak M A, Joshi P C. The nature and molecular basis of cutaneous photosensitivity reactions to psoralens and coal tar., J Invest Dermatol. 1983 June; 80 Suppl:66s-74s]. They are known to be involved in inflammation processes, cardiovascular disease [Hertog M G, Feskens E J, Hollman P C, Katan M B, Kromhout D. Dietary antioxidant flavonoids and risk of coronary heart disease: the Zutphen Elderly Study. Lancet. Oct. 23, 1993; 342(8878): 1007-11; Moure A. Franco D, Sineiro J, Dominguez H. N\dot{u}ñez M J, Lema J M. Evaluation of extracts from Gevuina avellana hulls as antioxidants. J Agric Food Chem. 2000 September; 48(9): 3890-7; Hollman P C, Katan M B. Health effects and bioavailability of dietary flavonols. Free Radic Res. 1999 December; 31 Suppl: S75-80.], rheumatoid arthritis, neurodegenerative disease, and the ageing process [Meyer T E, Liang H Q, Buckley A R, Buckley D J, Gout P W, Green E H, Bode A M. Changes in glutathione redox cycling and oxidative stress response in the malignant progression of NB2 lymphoma cells. Int J Cancer. Jul. 3, 1998; 77(1): 55-63; Hunt E J, Lester C E, Lester E A, Tackett R L. Effect of St. John's wort on free radical production. Life Sci. Jun. 1, 2001; 69(2): 181-90],

Antioxidants can prevent undesirable oxidation processes by reacting with free radicals, chelating free catalytic metals and also by acting as oxygen scavengers.

Antioxidants are present in foods as vitamins, minerals, carotenoids, and polyphenols, among others. Many antioxidants are often identified in food by their distinctive colors—the deep red of cherries and of tomatoes; the orange of carrots; the yellow of corn, mangos, and saffron; and the blue-purple of blueberries, blackberries, and grapes. The most well-known components of food with antioxidant activities are vitamins A, C, and E; β-carotene; the mineral selenium; and more recently, the compound lycopene. The research continues to grow regarding the knowledge of antioxidants as healthful components of food. Oxidation, or the loss of an electron, can sometimes produce reactive substances known as free radicals that can cause oxidative stress or damage to the cells. Antioxidants, by their very nature, are capable of stabilizing free radicals before they can react and cause harm, in much the same way that a buffer stabilizes an acid to maintain a normal pH. Because oxidation is a naturally occurring process within the body, a balance with antioxidants must exist to maintain health.

While the body has its defenses against oxidative stress, these defenses are thought to become less effective with aging as oxidative stress becomes greater [Knight, J A. The biochemistry of aging. Adv Clin Chem. 2000; 35:1-62]. Research suggests there is involvement of the resulting free radicals in a number of degenerative diseases associated with aging, such as cancer, cardiovascular disease, cognitive impairment, Alzheimer's disease, immune dysfunction, cataracts, and macular degeneration [McCall M R, Frei B. Can antioxidant vitamins materially reduce oxidative damage in humans? Free Radic Biol Med. 1999; 26; 7/8: 1034-53; Halliwell B. Oxygen and nitrogen are pro-carcinogens. Damage to DNA by reactive oxygen, chlorine and nitrogen species: measurement, mechanism and effects of nutrition. Mutat Res. 1999; 443: 37-52; Valko M, Izakovic M, Mazur M, Rhodes C J, Telser J. Role of oxygen radicals in DNA damage and cancer incidence. Mol Cell. 2004; 266: 37-56; Packer L, Weber S U, Rimbach G. Molecular aspects of α-tocotrienol antioxidant action and cell signaling. J Nutr. 2001; 131: 369S-373S; Aslan M, Ozben T. Reactive oxygen and nitrogen species in Alzheimer's disease. Curr Alzheimer Res. 2004; 1: 111-119; Ryan-Harshman M, Aldoori W. The relevance of selenium to immunity, cancer, and infectious/inflammatory diseases. Can J Diet Prac Res. 2005; 66: 98-102; Meyer C H, Sekundo W. Nutritional supplementation to prevent cataract formation. Dev Ophthalmol, 2005; 38: 103-119; Harman D. Nutritional implications of the free-radical theory of aging. J Am Coll Nutr. 1982; 1: 27-34]. Certain conditions, such as chronic diseases and aging, can tip the balance in favor of free radical formation, which can contribute to ill effects on health.

Consumption of antioxidants is thought to provide protection against oxidative damage and contribute to positive health benefits. For example, the carotenoids lutein and zeaxanthin engage in antioxidant activities that have been shown to increase macular pigment density in the eye. Whether this will prevent or reverse the progression of macular degeneration remains to be determined [Burke J D, Curran-Celentano J, Wenzel A J. Diet and serum carotenoid concentrations affect macular pigment optical density in adults 45 years and older. J Nutr. 2005; 135: 1208]. An increasing body of evidence suggests beneficial effects of the antioxidants present in grapes, cocoa, blueberries, and teas on cardiovascular health, Alzheimer's disease, and even reduction of the risk of some cancers [Fassina G, Vene R, Morini M, Minghelli S, Benelli R, Noonan D M, Albibi A. Mechanisms of inhibition of tumor angiogenesis and vascular tumor growth by epigallocatechin-3-gallate. Clin Cancer Res. 2004; 10: 4865-73; Rietveld A, Wiseman S. Antioxidant effects of tea: Evidence from human clinical trials. J Nutr. 2003; 13: 3285S-3292S; Rezai-Zadeh K, Shytle D, Sun N, Mori T, Hou H, Jeanniton D, Ehrhart J, Townsend K, Zeng J, Morgan D, Hardy J, Town T, Tan J. Green tea epigallocatechin-3-gallate (EGCG) modulates amyloid precursor protein cleavage and reduces cerebral amyloidosis in Alzheimer transgenic mice. J Neurosci. 2005; 25: 8807-8814; Lau F C, Shukit-Hale B, Joseph J A. The beneficial effects of fruit polyphenols on brain aging. Neurobiol Aging. 2005; Wiesburger J H. Lifestyle, health and disease prevention: the underlying mechanisms. Eur J Cancer Prev. 2002; S2: 1-7].

Until recently, it appeared that antioxidants were almost a panacea for continued good health. It is only as more research has probed on the mechanisms of antioxidant action that a far more complex story continues to be unraveled. Although recent research has attempted to establish a causal link between indicators of oxidative stress and chronic disease, none has yet been validated. A new area of research, led by the study of the human genome, suggests that the interplay of human genetics and diet may play a role in the development of chronic diseases. This science, while still in its infancy, seeks to provide an understanding of how common dietary nutrients such as antioxidants can affect health through gene-nutrient interactions [Kaput J, Ordovas J M, Ferguson L, Ommen B V, Rodriquez R, Allen L, Ames B, Dawson K, German B, Krauss R, Malyj W. The case for strategic international alliances to harness nutritional genomics for public and personal health. Br J Nutr. 2005; 94: 623-632].

There still remains a lack of direct experimental evidence from randomized trials that antioxidants are beneficial to health, which has led to different recommendations for different populations. For example, the use of supplemental β-carotene has been identified as a contributing factor to increased risk of lung cancer in smokers [Goodman G E, Thornquist M D, Balmes J, Cullen M R, Meyskens F L Jr, Omenn G S, Valanis B, Williams J H Jr. The β-Carotene and Retinol Efficacy Trial: incidence of lung cancer and cardiovascular disease mortality during 6-year follow-up after stopping β-carotene and retinol supplements, J Natl Cancer Inst. 2004; 96: 1743-1750].

However, because the risk has not been indicated in non-smokers, these studies suggest that a precaution regarding the use of supplemental β-carotene is not warranted for non-smokers. If supplementation is desired, the use of a daily multivitamin-mineral supplement containing antioxidants has been recommended for the general public as the best advice at this time [Fairfield K, Fletcher R. Vitamins for Chronic Disease Prevention in Adults: Clinical Applications. JAMA. 2002; 287: 3127-3129].

A recent review of current literature suggests that fruits and vegetables in combination have synergistic effects on antioxidant activities leading to greater reduction in risk of chronic disease, specifically for cancer and heart disease [Liu R H, Potential Synergy of Phytochemicals in Cancer Prevention: Mechanism of Action. J. Nutr. 2004; 134: 3479S-3485S].

For some time, health organizations have recognized the beneficial roles fruits and vegetables play in reducing the risk of diseases, and developed communication programs to encourage consumers to eat more antioxidant-rich fruits and vegetables. The American Heart Association recommends healthy adults “Eat a variety of fruits and vegetables. Choose 5 or more servings per day” [Krauss R M, Eckel R H, Howard B, Appel L J, Daniels S R, Deckelbaum R J, Erdman J W, Etherton P K, Goldberg I J, Kotchen T A, Lichtenstein A H, Mitch W E, Mullis R, Robinson K, Wylie-Rosett J, St. Jeor S, Suttie J, Tribble D L, Bazzarre T L. AHA Dietary Guidelines Revision 2000: A statement for healthcare professionals from the nutrition committee of the American Heart Association. Circulation. Available at: http://circ.ahajournals.org/cgi/content/full/4304635102].

The American Cancer Society recommends to “Eat 5 or more servings of fruits and vegetables each day .” [ACS Recommendations for Nutrition and Physical Activity for Cancer. Available at: http://www.cancer.org/docroot/PED/content/PED_{—}3_{—}2X_Recommendations.asp?siteara=PED. The World Cancer Research Fund and the American Institute for Cancer Research 1997 Report Food, Nutrition and the Prevention of Cancer: A Global Perspective states, “Evidence of dietary protection against cancer is strongest and most consistent for diets high in vegetables and fruits” [World Cancer Research Fund International—Food, Nutrition and the Prevention of Cancer: a global perspective. Available at: http://www.wcrf.org/research/fnatpoc.lasso]. The potential for antioxidant-rich fruits and vegetables to help improve the health of Americans led the National Cancer Institute (NCI) to start the, “5-A-Day for Better Health” campaign to promote consumption of these foods [Heimendinger J, Stables G, Foerster S. The Scientific Policy and Theoretical Foundations for the National 5 A Day for Better Health Program. Available at: http://5aday.gov/about/pdf/5aday_ch1.pdf].

Given the high degree of scientific consensus about consumption of a diet that is high in fruits and vegetables—particularly those which contain dietary fiber and vitamins A and C; the Food and Drug Administration (FDA) released a health claim for fruits and vegetables in relation to cancer. Food packages that meet FDA criteria may now carry the claim “Diets low in fat and high in fruits and vegetables may reduce the risk of some cancers” [Food and Drug Administration—Center for Food Safety and Applied Nutrition Code of Federal Regulations: Title 21, V 2. Available at: http://www.cfsan.fda.gov/˜lrd/cf101-78.html]. In addition the FDA, in cooperation with National Cancer Institute (US Government), released a dietary guidance message for consumers, “Diets rich in fruits and vegetables may reduce the risk of some types of cancer and other chronic diseases” [Food and Drug Administration—Center for Food Safety and Applied Nutrition Dietary Message about Fruits and Vegetables: Available at: http://www.cfsan.fda.gov/˜dms/lab-dg.html]. Most recently the Dietary Guidelines for Americans stated, “Increased intakes of fruits, vegetables, whole grains and fat-free or low-fat milk and milk products are likely to have important health benefits for most Americans” [U.S. Department of Health and Human Services, U.S. Department of Agriculture. Dietary Guidelines for Americans 2005. 6th ed., Washington, D.C.: U.S. Government Printing Office; 2005]. Antioxidant research continues to grow and emerge as new beneficial components of food are discovered. Reinforced by current research, the message remains that antioxidants obtained from food sources, including fruits, vegetables and whole grains, are potentially active in disease risk reduction and can be beneficial to human health [Tribble D L. Antioxidant consumption and risk of coronary heart disease: Emphasis on vitamin C, vitamin E and β-carotene. Circulation, 1999; 99: 591-595]. Currently, there are over 500 clinical trials (http://clinicaltrials.gov/ct/) organized by the US Government to find newer therapeutic uses of antioxidants.

In addition to the above health benefits of antioxidants, a significant usefulness comes from their use in protecting foods and crops from spoiling. Fruits and vegetables treated with antioxidants can be stored for a longer period of time and this becomes particularly important when the crops are shipped across the globe.

Antioxidants are also widely used to protect the oxidazable surfaces such as metallic surfaces; this can be particularly critical where the rusting of metals can result in poor circuit contacts and cause failure of equipment.

Antioxidants are also an essential component of various pharmaceutical formulations and food products that contain fats that are likely to undergo oxidation leading to rancidity; butylated hydroxy anisole and butylated hydroxy toluene are the most commonly used examples but compounds like vitamin C and vitamin E or their derivatives are also widely used.

As a result of the numerous benefits of antioxidants, a large number of potential antioxidants have been synthesized, extracted or otherwise specifically designed. However, for an antioxidant to be effective it must pass the safety and efficacy requirements pursuant to the application before it is used. This invention reports extremely safe and effective antioxidants from lichens which contain a variety of compounds comprising of simple aromatics, depsides, depsidones, dibenzofurans, and triterpenoids, that can prove to be potent antioxidants as evidenced by the their antioxidant activity using superoxide radical scavenging assay (SOR).

Lichens are small perennial plants consisting of a symbiotic association of a fungus and an alga. They produce characteristic secondary metabolites that are unique with respect to those of higher plants. Several lichen extracts have been used for various remedies in folk medicine, and screening tests have indicated lichens as unique organisms producing biologically active metabolites with a great variety of effects such as antibiotic [Boustie J, and Grube M. Lichens—a promising source of bioactive secondary metabolites. Plant Genetic Resources. 2005; 3: 273-287], anti-mycobacterial, [Ingolfsdottir K, Chung G A C, Skulason V G, Gissurarson S R, Vilhelmsdottir M. Antimicobacterial activity of lichen metabolites invitro. Eur. J. Pharm. Sci. 1998; 6: 141-144; Muller K. Antimicobacterial pharmaceutically relevant metabolites from lichens. Applied Microbiology and Biotechnology 2001; 56(1-2): 9-16], antiviral [Yamamoto Y, Miura Y, Kinoshita Y, Higuchi M, Yamada Y, Murakami A, Ohigashi H, Koshimizu K. Antimicrobial, antiviral, and cytotoxic activity of Newzealand lichens. Chem. Pharm. Bull. 1995; 43: 1388-1390; Neamati N, Hong H, Mazumder A, Wang S, Sunder S, Nicklaus M C, Milne G W, Proksa B, Pommier Y. Depsides and depsidones as inhibitors of HIV-1 integrase: discovery of novel inhibitors through 3D database searching. J. Med. Chem. 1997; 40: 942-951], analgesic and antipyretic properties [Okuyama E, Umeyama K, Yamazaki M, Kinoshita Y, Yamamoto Y. Usinic acid and diffractaic acid as analgesic and antipyretic compounds of Usnea diffracta. Planta Med. 1995; 61; 113-115].

However, only very limited numbers of lichen substances have been screened for their biological activities and their therapeutic potential in medicine. This may partly be due to the difficulties encountered in identification of the species, and collecting substantial amounts of plant material, as most of the lichen species grow as scattered patches, mainly on stones or on tree trunks. The study of bioactivities of lichen compounds is important because the secondary metabolites of lichens are found almost exclusively only in lichens. Out of the approximately 800 secondary metabolites known up to 80% are restricted to the lichenized state [Huneck, S. and Yoshimura, I. Identification of Lichen substances 1996, Springer-Verlag].

Many lichens grow under erratic and extreme conditions of temperature, humidity, and intensity of light where the stress induced results in unpredictable synthesis of metabolites [Caviglia A M, Nicora P, Giordani P, Brunialti G, and Modenesi. Oxidative stress and usnic acid content in Parmelia caperata and Parmelia soredians (Lichens). IL Farmac. 2001; 56: 379-382]. The crustose species Pertusaria alaianta Nyl., from the Cape Verde Islands, for example, in hot and arid climate contains up to 20% dry weight of a mixture of chloroxanthones. Such high amounts of secondary compounds can hardly be found in higher plants [Huneck, S. and Yoshimura, I. Identification of Lichen substances 1996, Springer-Verlag. Interestingly, these metabolites are very stable demonstrating shelf-lives of over 100 years as evidenced by herbarium specimens of lichens. In brief, the structure-activity relationship of lichen compounds can be unpredictable and forms the basis of our surprising findings of highly active antioxidants that can be used in a variety of commercial applications.

### Experimental

### Isolation and Identification of Lichen Metabolites

Cleaned, dried lichens were sequentially extracted with methylene chloride followed by methanol. The crude methylene chloride extract and methanol extract were fractionated via silica gel Medium Pressure Liquid Chromatography (MPLC) using accelerating gradient elution with a SEPARO column packed with Merck Kieselgel (230-400 mesh ASTM) and metering pump FM1-pump, model QD OSSY and column chromatography. Lichen compounds were separated by using the combinations of hexane-methylene chloride or hexane-ethyl acetate or dichloromethane-methanol in stepwise gradients. Identity of the known compounds was determined by comparison of the physical data (thin layer chromatography, co-thin layer chromatography and melting point) of the isolated compounds with those of the authentic samples and reported spectral data [¹D and ²D Nuclear Magnetic Resonance (NMR) spectra and Mass Spectra (MS)]. Analytical thin layer chromatography was carried out on Kieselgel 60 pre-coated aluminum foil plates. The spots on the thin layer chromatography plates were detected under UV light (wavelength 254 and 365 nm) and spraying with anisaldehyde. ¹H and ¹³C NMR, Correlation Spectroscopy (COSY), Distortion Enhanced Polarization Transfer (DEPT), Heteronuclear Correlation Spectroscopy (HETCOR), Heteronuclear Multiple Quantum Correlation (HMQC), Heteronuclear Multiple Bond Correlation (HMBC) and Nuclear Overhauser Enhancement Spectroscopy (NOESY) spectra were recorded on a VARIAN 300 MHz machine at ambient temperature at 30° C. Electron Spray Ionization Mass Spectroscopy (ESIMS) were recorded on a Fisions VG Autospec mass spectrometer operating at 70 eV (direct insertion). High Resolution Electron Spray Ionization Mass Spectroscopy (HRESIMS) were recorded on a Micromass LCT spectrometer. Arg-Lys, perfluorokerosine and Arg-Phe were used as the internal reference for HRMS measurements. Purity of the compounds were confirmed using analytical high pressure liquid chromatography using Waters 2690 pump coupled to ultraviolet photodiode array detector Waters 996 using a Novapack C₁ₛ reversed phase column, and methanol and water as eluents.

Four potent and novel antioxidants were discovered and identified using the techniques described above. These included lecanoric acid (Compound I) obtained from Parmotrema grayana Hue using methanolic extraction, erythrin (Compound II) from Rocella montagnei Bel using acetone extraction, sekikaic acid (Compound III) from Heterodermia obscurata (Nyl.) Trevisan using methanolic extraction and lobaric acid (Compound IV) from Cladonia sp., using methanolic extraction.

### Isolation of Active Antioxidants

More specifically, the methanol extract of Parmotrema grayana when subjected to Medium Pressure Liquid Chromatography (MPLC) (hexane/methylene chloride to methylene chloride/methanol) and refractionated via MPLC (hexane to ethyl acetate) and recrystalizing using ethyl acetate/hexane afforded lecanoric acid [FIG. 1: Compound I, (CAS: 607-11-4), which is a para depside (methylated derivatives: 537-09-07, 3542-22-1, 70342-21-10, 4382-39-2, 107783-44-8)]in 3.2% yield. The acetone extract of Rocella montagnei when subjected to MPLC (hexane/methylene chloride/methanol) and re-subjected to MPLC again using methylene chloride to methylene chloride/methanol gave erythrin (FIG. 2: Compound II CAS: 480-57-9, a para depside) in 7.3% yield; the methanolic extract of Heterodermia obscurata when subjected to MPLC (hexane/methylene chloride to methylene chloride/methanol) and refractionated via MPLC (20% hexane/methylene chloride to 20% methanol/methylene chloride), and gravity chromatography (5% hexane/methylene chloride to 10% methanol/methylene chloride) afforded, on recrystalization (1% methanol/methylene chloride), the depside, sekikaic acid [FIG. 3: Compound III, CAS: 607-11-4, a meta depside, (methylated derivatives 69563-42-4, 73694-32-3, 15081-04-6, 103538-07-4, 103538-08-5, 69563-43-5)] in 1.07% yield; and the methanolic extract Cladonia sp. when subjected to MPLC (hexane/methylene chloride to methylene chloride/methanol) and subjected again to MPLC (hexane/methylene chloride to methanol/methylene chloride), it afforded the depsidone lobaric acid [(FIG. 4: Compound IV, CAS: 522-53-2, a depsidone, (methylated derivatives 29813-50-1, 20661-49-8 and de-methylated derivative 29813-65-8)] on recrystallization (97% methylene chloride/methanol) in 0.37% yield.

### BRIEF DESCRIPTION OF THE DRAWING

FIG. 1: Chemical Structure of lecanoric acid (Compound I)

FIG. 2: Chemical Structure of erythrin (Compound II)

FIG. 3: Chemical Structure of sekikaic acid (Compound III)

FIG. 4: Chemical Structure of lobaric acid (Compound IV)

FIG. 5: Common structural feature of SOR

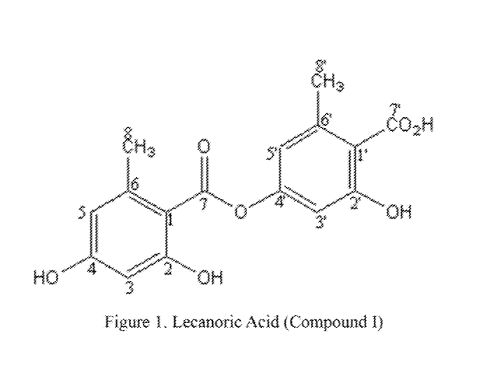

FIG. 1

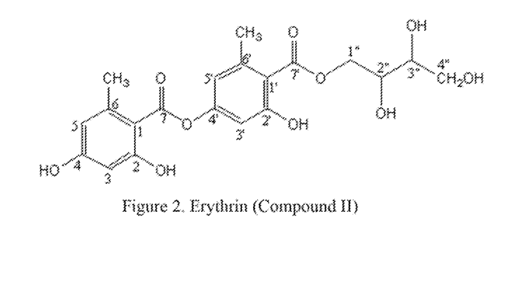

FIG. 2

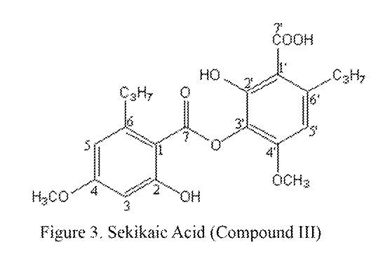

FIG. 3

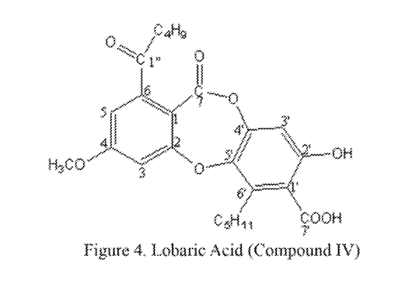

FIG. 4

Additional studies were carried out using the permethylated derivates of lecanoric acid and erythrin. Lecanoric acid (500 mg) or erythrin (500 mg), and 500 mg of anhydrous potassium carbonate were dissolved in dimethyl sulfoxide (25 ml), and to this mixture methyl iodide (0.25 ml) was added and stirred at 25° C. under anhydrous conditions for 3 hours. The reaction mixture was then acidified with cold dilute hydrochloric acid and extracted into ethyl acetate. The combined organic fractions were washed with several portions of water, dried magnesium sulfate and the solvent was removed to give the crude product which was purified via silica gel MPLC (gradient eluant: hexane/methylene chloride to methylene chloride/methanol) to afford the permethylated depsides of lecanoric acid and erythrin respectively.

### Antioxidant Assays

The reaction mixture contained 10 μL of test samples (1 mM in dimethylsulfoxide), 90 μL of 0.1 M phosphate buffer (pH 7.4), 40 μL of (280 μM) β-nicotanamide adenine dinucleotide (NADH), 40 μL of (80 μM) nitro blue tetrazolium (NBT). The reaction was initiated by the addition of 20 μL of (8 μM) phenazine methosulphate (PMS). The solutions of NADH, NBT and PMS were prepared in phosphate buffer. The formation of superoxide was monitored by measuring the absorbance of the blue formazan dye after three minutes at 560 nm against the corresponding blank solutions in microtitre plate using Elisa (multiple reader spectra Max-3400). IC₅₀ value represents concentration of compounds needed to scavenge 50% of super oxide radicals. Propyl gallate was used as a positive control. All chemicals used were of analytical grade (Sigma Chemicals, USA).

### Results

The antioxidant activity of various lichen extracts has been reported [Behera B C, Verma N, Sonone A, and Makhija U. Antioxidant and antibacterial activities of Usnea ghattensis in vitro. Biotechnology Letters, 2005; 27: 991-995; Behera B C, Adawadkar B, and Makhija U. Tissue culture of selected species of Graphis lichen and their biological activities. Fitoterapia. 2006; 77: 208-215; Halici M, Odabasoglu F, Suleyman H, Cakir A, Aslan A, and Bayir Y. Effects of water extracts of Usnea longissima on antioxidant enzyme activity and mucosal damage caused by indomethacin in rats. Phytomedicine. 2005; 12: 656-662; Jayaprakasha G K, and Rao L J. Phenolic constituents from the lichen Parmotrema stuppeum (Nyl.) Hale and their antioxidant activity. Z. Naturforsh. 2000; 5c: 1018-1022]. These studies revealed that there was a correlation between the total phenols in the extracts and the antioxidant activity suggesting that the antioxidant activity was probably due to phenolic compounds. However, prior to this invention, there have been no reports in the literature of other prior art on the antioxidant activity of the pure lichen substances, particularly Compounds I-IV.

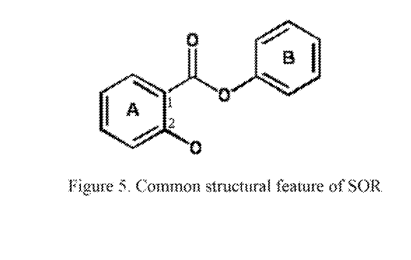

The para depsides lecanoric acid (Compound I), erythrin (Compound II) and the meta depside sekikaic acid (Compound III) showed exceptionally high percentage of radical scavenging activity in the SOR assay along with the depsidone lobaric acid (Compound IV). The common structural feature in all of the above compounds, is two aromatic rings connected by an ester linkage, and ortho to the carbonyl bearing carbon of ring A, an oxygen atom which may act as the electron acceptor from the antibonding orbitals of superoxide radical leading to molecular oxygen (FIG. 5). The electron thus obtained could be stabilized due to extended conjugation available in such compounds. In the case of depsidone lobaric acid (Compound IV), the electron accepted by C-2-O could be stabilized by both aromatic rings.

FIG. 5

The SOR activity of both the depsides lecanoric acid (Compound I) and erythrin (Compound II) were lost on per-methylation suggesting that when C-2-O is methylated the molecule looses its ability to accept electrons. Importantly, the IC₅₀ values of the sekikaic acid (Compound III) lecanoric acid (Compound I), and lobaric acid (Compound IV) were lower than the propyl gallate standard (Table 1).

TABLE 1
IC₅₀ values of the active compounds of super oxide scavenging assay

| Compound | SOI (IC₅₀ ± SEM) |
|---|---|
| Lecanoric acid (Compound I | 91.45 ± 2.10 |
| Erythrin (Compound II) | 127.04 ± 0.97 |
| Sekikiac acid (Compound III) | 81.97 ± 0.31 |
| Lobaric acid (Compound IV) | 97.94 ± 1.60 |
| Standard (propyl gallate) | 106 ± 1.70 |
| Standard (BHA) | 96.0 ± 1.75 |

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A novel antioxidant composition comprising of an effective amount of lecanoric acid and optionally a suitable carrier for use in humans, animals, foods and crops.

13. A novel antioxidant composition comprising of an effective amount of erythrin, and optionally a suitable carrier for use in humans, animals, foods and crops.

14. A novel antioxidant composition comprising of an effective amount of sekikaic acid and optionally a suitable carrier for use in humans, animals, foods and crops.

15. A novel antioxidant composition comprising of an effective amount of lobaric acid and optionally a suitable carrier for use in humans, animals, foods and crops.

16. The composition of claim 12-15 wherein said composition is used to treat cancer, cardiovascular disease, rheumatoid arthritis, cystic fibrosis, ageing process, degenerative diseases, cataracts, alopecia, and other disorders triggered by the presence of excessive free radicals in the body.

17. The composition of claims 12-15 wherein said composition is used to protect crops, fruits and vegetables from spoiling and decaying due to oxidation.

18. The composition of claims 12-15 wherein said composition is used to protect surfaces against oxidation.

19. The composition of claims 12-15 wherein said composition is used to protect degradation of oily components in foods and drugs.

20. The composition of claims 12-15 wherein said compounds are obtained from a natural source by a process of extraction of plants or plant parts, more specifically, lichens.

21. The composition of claim 12-15 wherein said composition additionally contains other known antioxidants, free-radical scavengers, and metal chelants.
